(19) United States
(12) Patent Application Publication
(10) Pub. No.: US 2007/0260298 A1
(43) Pub. Date: Nov. 8, 2007

(54) Pulsed-light electric medical appliance for skin treatment

(76) Inventor: Moreno Naldoni, Scandicci (IT)

Correspondence Address: LADAS & PARRY, 26 WEST 61ST STREET, NEW YORK, NY, 10023, US

(21) Appl. No.: 11/332,601

(22) Filed: Jan. 11, 2006

Related U.S. Application Data
(63) Continuation of application No. PCT/IT02/00737, filed on Nov. 20, 2002.

(30) Foreign Application Priority Data
Nov. 21, 2001 (IT) BO01A000706

(51) Int. Cl. A61N 5/06

(52) U.S. Cl. 607/94

(57) ABSTRACT

The appliance (1) has a handset (5), in turn having a lamp (13) for generating pulsed light onto a patient's skin, and an electric circuit (14) for starting the lamp (13). The appliance also has a box body (11) housing the lamp (13) and the electric circuit (14); and means (38, 41) for supporting the box body (11) so that it can be removed easily from the handset (5).

### TECHNICAL FIELD

The present invention relates to a pulsed-light electric medical appliance for skin treatment, and in particular for removing unwanted hair, reducing blotches, rejuvenating the skin, and vascular treatment.

### BACKGROUND ART

As is known, appliances of the above type provide for directing onto the skin area for treatment high-energy pulsed light in the ultraviolet to intermediate infrared wavelength range, and comprise a machine body housing the electric and electronic devices for producing the necessary light-generating energy; pulsed-light control electronics; and a handset outside the machine body, and which is applied to the skin and houses the light source defined by a gas or incandescent lamp. The handset is preferably fitted with one or more filters, made of glass or other suitable material, for cutting off given undesired wavelengths.

Appliances of the above type have several drawbacks. In particular, the lamp produces heat which may exceed 400° C., and which must be removed to prevent premature wear of certain parts of the handset, and to safeguard the patient against burns caused by contact with such parts. Moreover, the light applied to the skin penetrates the tissue and superheats the hair bulb, but also burns the hair on the outside of the skin, thus resulting in smoke which must be disposed of. Conventional appliances feature complex lamp cooling systems (Peltier cells, water, etc.) and equally complex skin cooling systems (Peltier cell rings or plates, or water); and handset-skin interface gel must also be used. As a result, the handsets are heavy, unwieldy, and complex to make. If no cooling systems are provided, the handsets may be fairly straightforward and easy to manage, but only at the expense of forgoing high power and separate optical filters, which, operating in contact with the skin and directly facing the power lamp, are subject to severe heating and a frequent cause of burns.

Moreover, fast-wear component parts are difficult to change, by either being housed inside the machine body, or forming part of a complex, high-cost assembly.

Some appliances feature a fast-change lamp, which may have a filter coating, but do not allow for using separate optical filters, and have a low power level.

### DISCLOSURE OF THE INVENTION

It is an object of the present invention to provide an electric medical appliance for skin treatment, designed to eliminate the aforementioned drawbacks, and which generates high power levels without forgoing the use of separate optical filters—extremely useful for adapting treatment to the patient's skin type. At the same time, the appliance according to the present invention features an extremely straightforward, low-cost air cooling system, and an innovative, compact, easy-change box assembly housing all the component parts subject to wear.

More specifically, the handset of the appliance according to the present invention comprises a grip; a head portion; and a box which is fitted and removed easily to and from the head portion, and houses the optical filter and electronic components requiring frequent replacement.

The box comprises a filter seat at such a distance from the skin as to prevent contact between the skin and filter; and a number of openings located at a rim of the box, and for cooling the inside of the box by outside air flowing through the gap between the optical filter and lamp.

According to the present invention, there is provided an electric medical appliance for skin treatment, of the type comprising a handset housing a lamp for generating, onto an area of a patient's skin, high-energy pulsed light in an ultraviolet to intermediate infrared wavelength range, and an electric circuit for starting said lamp; characterized by comprising, inside a head portion of the handset, a box body housing said lamp and said electric circuit; the handset also containing means for supporting and enabling withdrawal of said box body from the head portion.

In addition to the lamp and relative start circuit, the box body also houses one or more superimposed filters, each defined by a sheet of glass and for cutting off light of given wavelengths. The present invention is characterized in that the box body housed inside said handset comprises an application window which is placed on the skin; and a support for said filters, which is located slightly higher than said window and formed in the walls of the box body.

### BRIEF DESCRIPTION OF THE DRAWINGS

A non-limiting embodiment of the present invention will be described by way of example with reference to the accompanying drawings, in which:

FIG. 1 shows a view in perspective of an electric medical appliance for skin treatment, in accordance with the teachings of the present invention;

FIG. 2 shows a larger-scale view in perspective of part of a handset of the FIG. 1 appliance;

FIG. 3 shows a longitudinal scale section of the FIG. 1 appliance handset;

FIG. 4 shows a cross section of a head portion of the FIG. 3 handset;

FIG. 5 shows an exploded view in perspective of the FIG. 3 handset.

### BEST MODE FOR CARRYING OUT THE INVENTION

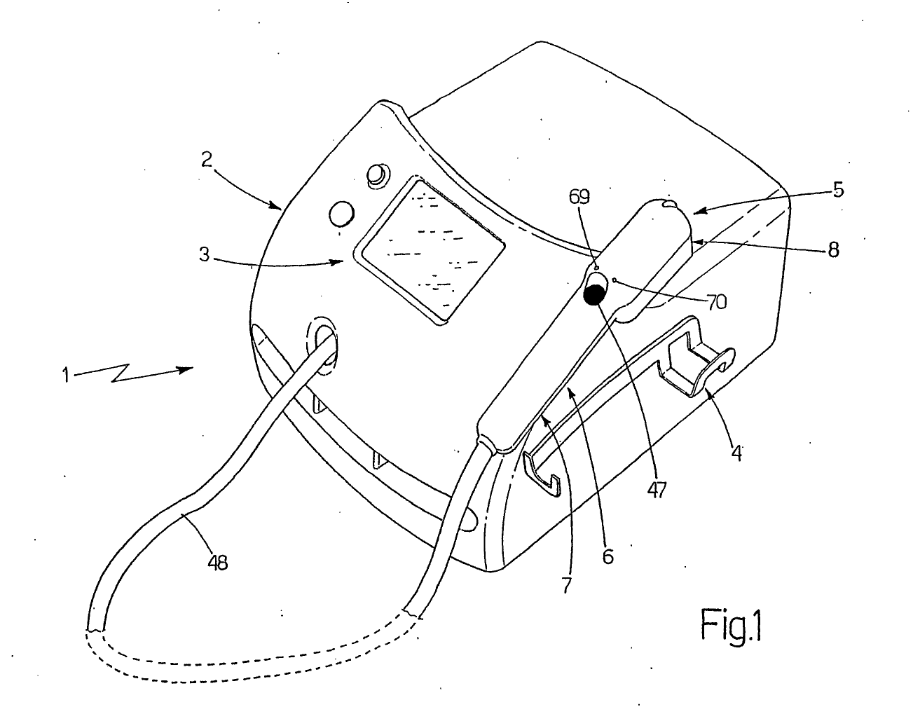

Number 1 in FIG. 1 indicates as a whole a pulsed-light electric medical appliance for skin treatment, which may be used for removing unwanted hair, reducing blotches, rejuvenating the skin, vascular treatment, and other commonly practised applications, and which, as is known, provides for directing, onto the skin area for treatment, high-energy pulsed light in the ultraviolet to intermediate infrared wavelength range.

With reference to FIG. 1, appliance 1 comprises a machine body 2 in which are housed an air intake pump; an electric power block; a block of discharge capacitors and relative control circuits; and a microprocessor block for controlling appliance 1 as a whole. All the above components inside machine body 2 are known types and therefore not shown. Machine body 2 supports a known—in particular, touch screen—control panel 3 having a number of touch buttons for controlling the above components, and a display showing treatment cycle stages and the values of various physical quantities involved; and a seat 4 for a handset 5 having a number of innovative characteristics as explained below.

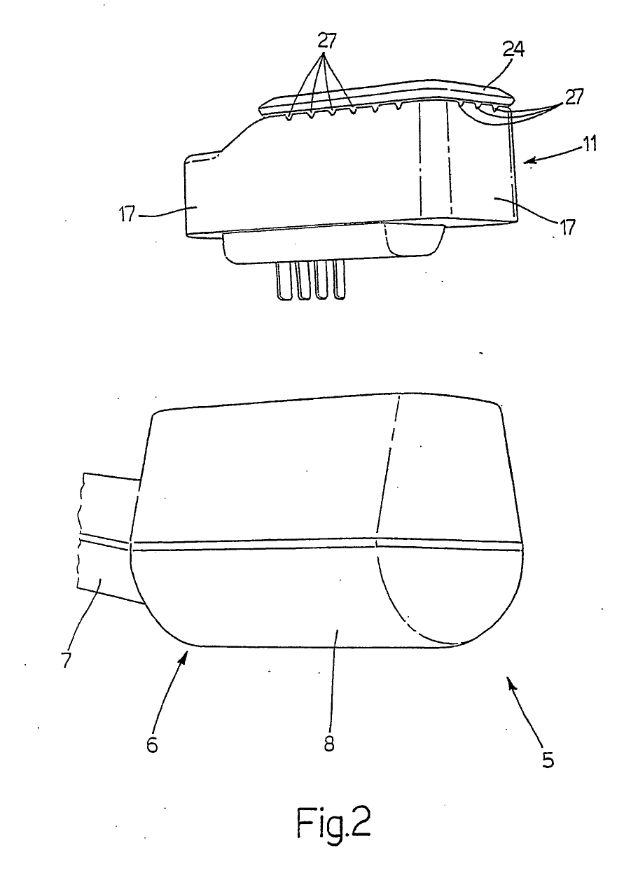

With reference to FIGS. 2 to 5, handset 5 comprises a shell 6 preferably made of plastic material in two parts 6a and 6b connected integrally by screws 6c. Shell 6 is defined by a grip 7 and a head portion 8, and handset 5 also comprises a box body 11 housed partly inside head portion 8 through an opening 12 in portion 8. Box body 11 houses a preferably U-shaped xenon lamp 13; an electric circuit 14 for starting lamp 13; and a filter 15 for cutting off light of wavelengths outside a given range, lamp 13 being a source of light in the ultraviolet to intermediate infrared wavelength range.

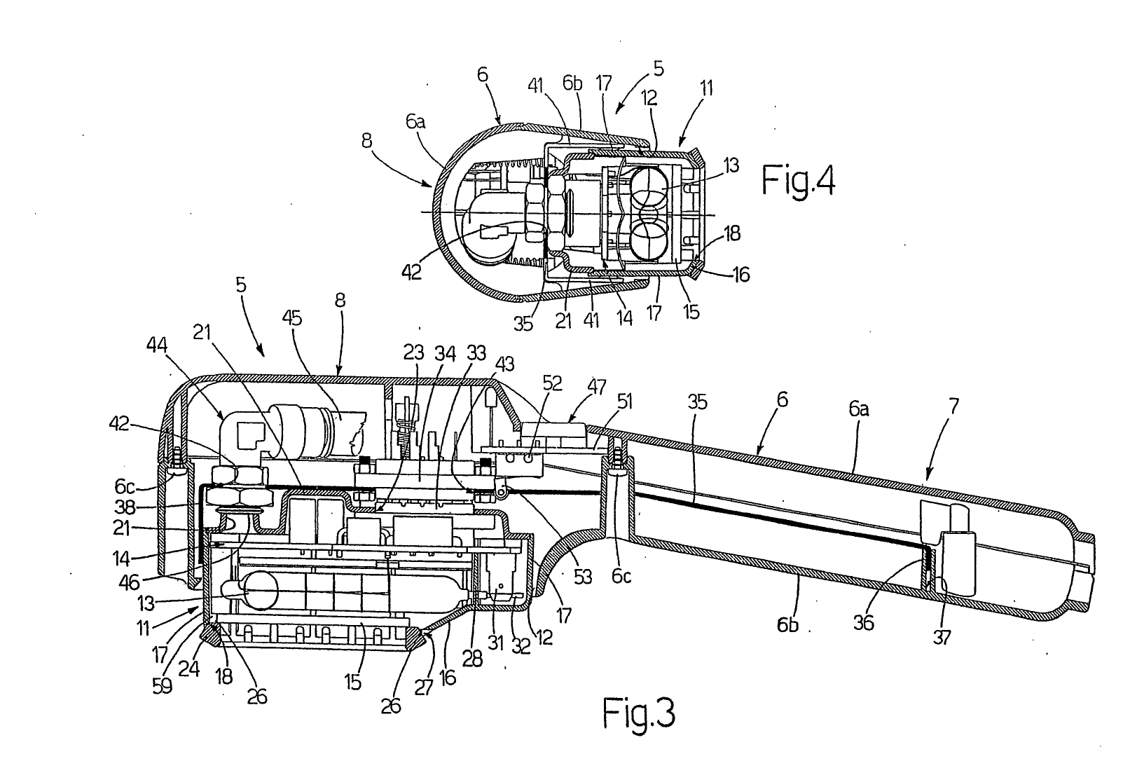

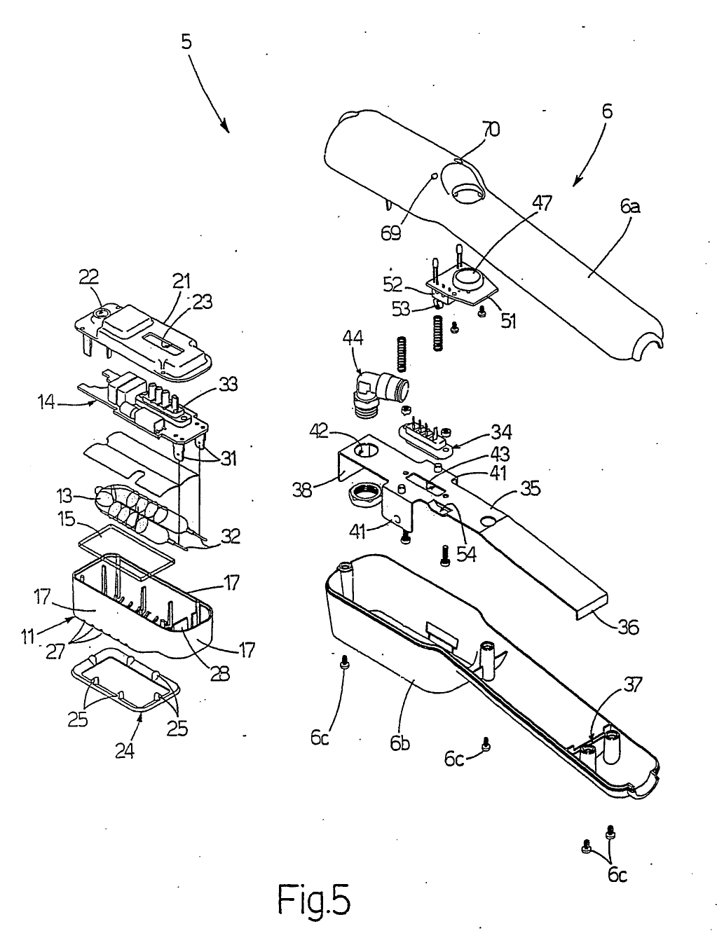

With reference to FIGS. 3 to 5, box body 11 comprises a base wall 16 and four lateral walls 17; a rectangular window 18 is formed in base wall 16; and box body 11 is closed at the top by a cover 21 in which are formed a through hole 22 and a through slot 23. At window 18, box body 11 is fitted inside with filter 15, which is defined by a sheet of glass fixed to a support 59 formed in lateral walls 17. A rim 24, fitted to window 18, has teeth 25 (FIG. 5) which click inside appropriate seats 26 (FIG. 3) formed along the edge of window 18; rim 24 is of such a thickness that its bottom edge is outside box body 11; and a number of air inlets 27 are formed along the edge of window 18 to allow the inside of box body 11 to communicate with the outside.

With reference to FIGS. 3 to 5, a wall 28 for supporting lamp 13 extends from base wall 16 inside box body 11. Start circuit 14 is installed between lamp 13 and cover 21, and is defined by a printed circuit having electric terminals 31 soldered to the electric terminals 32 of lamp 13, and an electric plug 33 for connection, through slot 23, to an electric socket 34 in head portion 8 of handset 5.

With reference to FIGS. 3 to 5, handset 5 is fitted inside with a bracket defined by a metal plate 35, a first axial end 36 of which is bent and engages a seat 37 in grip 7, and a second axial end 38 of which is bent and, together with two bent lateral tabs 41, encloses box body 11 inside head portion 8 of handset 5. That is, box body 11 is supported by end 38 and tabs 41 of plate 35, so that, together with plate 35 being elastically deformable, box body 11 can be positioned with a certain amount of slack inside head portion 8 of handset 5.

With reference to FIGS. 3 to 5, the part of plate 35 housed in head portion 8 comprises a through hole 42 coaxial with hole 22; and a slot 43 coaxial with slot 23. More specifically, at hole 42 is fixed a first end of a pneumatic fitting 44, a second end of which supports a tube 45 (shown partly for simplicity) connected pneumatically to the pump housed in machine body 2. By means of a seal 46 (FIG. 3), the first end of fitting 44 is connected to hole 22, which has a raised edge; and socket 34 is fixed to plate 35 at slot 43.

With reference to FIGS. 3 and 5, head portion 8 of handset 5 comprises an electric pushbutton 47 operated, obviously, from the outside to electrically supply start circuit 14. More specifically, electric cables (not shown) extend from said electric power, discharge capacitor, and microprocessor blocks housed in machine body 2, and, outside machine body 2, are enclosed, together with tube 45, inside a sheath 48 (FIG. 1), one end of which is fitted to the axial end of grip 7 so that tube 45 is connected to fitting 44, the electric cables from the microprocessor block are connected to corresponding contacts on socket 34, and the electric cables from the power and discharge capacitor blocks are connected to corresponding contacts on socket 34 by means of pushbutton 47.

With reference to FIGS. 3 and 5, head portion 8 is fitted inside with a small electric circuit 51 which on one side (FIG. 1) supports pushbutton 47 and two LED's—a green LED 69 indicating the machine is ready, and a red LED 70 indicating the machine is charging—and on the opposite side supports a microswitch 52 electrically in series with pushbutton 47. Microswitch 52 has a pin 53 which is movable between a withdrawn position making the circuit electrically supplying circuit 14, obviously when pushbutton 47 is pressed, and an extracted position in which it breaks the supply circuit even when pushbutton 47 is pressed. Pin 53, or rather its outer axial end, cooperates with a recess 54 (FIG. 5) formed in plate 35. In actual use, when the bottom edge of rim 24 is placed on the patient's skin, even with pushbutton 47 pressed, circuit 14 is not powered; whereas, when rim 24 is pressed with a certain amount of force on the skin, box body 11 withdraws inside head portion 8 and against plate 35 sufficiently to withdraw pin 53 by means of plate 35 and so power circuit 14 with pushbutton 47 pressed.

In actual use, after turning on a main switch on control panel 3, rim 24 is placed on the patient's skin, is then pressed with a certain amount of force on the skin, as described above, and pushbutton 47 is pressed to supply start circuit 14 and commence treatment. This system prevents light flashing in the air, which may cause severe eye injury to anyone in the vicinity of appliance 1. The air flowing through air inlets 27 towards the pump is first channeled into box body 11, flows over the inner face of filter 15 and lamp 13, and is channeled out of body 11 through fitting 44, so as to effectively remove the hot air produced by the powerful light pulses, and, by cooling lamp 13 and optical filter 15, cool the inside of body 11 to prevent overheating of the components inside, and remove any smoke produced during treatment. The microprocessor control electronics inside machine body 2 provide for maintaining airflow for a given software-set time period, even with the machine off, provided it is connected to the electricity mains. This feature, not to be found in any similar currently marketed appliances, provides for more effective cooling of lamp 13, optical filter 15, and the other components in box body 11, by also continuing cooling during downtime between treatments and whenever appliance 1 is not in use.

The advantages of the present invention will be clear from the foregoing description.

In particular, an appliance is provided comprising, in the handset, a box body housing all the components normally subject to wear and so requiring frequent replacement. Moreover, the box body is easy to assemble and disassemble to and from the handset, by being connected to it simply by means of a click-on fastener and an electric connector, and has a cooling system which not only prevents overheating of said components but also keeps the optical filter clean. Another innovative feature is the given distance maintained between the filter and skin, thus safeguarding the skin against burns, and, together with the cooling airflow described above, enabling other lamp and skin cooling systems to be dispensed with. Finally, the handset is provided with a safety system, as described above, which only enables treatment to commence when the handset is pressed with a certain amount of force on the skin.

1. A pulsed-light electric medical appliance for skin treatment, of the type comprising a handset (5) housing a lamp (13) for generating, onto an area of a patient's skin, high-energy pulsed light in an ultraviolet to intermediate infrared wavelength range, and an electric start circuit (14) for starting said lamp (13); the appliance being characterized by comprising, inside the handset (5), a box body (11) housing said lamp (13), the relative electric start circuit (14), and an optical filter (15); and means (38, 41) for supporting said box body (11); the box body (11) being simply pulled out of and pressed into said handset (5) in one piece.

2. An appliance as claimed in claim 1, characterized in that said optical filter (15) is located on a support (59) formed in walls (17) of said box body (11), so as to be located an appropriate distance from, and not contact, the skin during treatment.

3. An appliance as claimed in claim 2, characterized in that said handset (5) comprises means (44, 45) for drawing air into said box body (11); the airflow being so channeled as to flow along the gap between the lamp (13) and the optical filter (15), so that other systems for cooling the lamp and skin are not required.

4. An appliance as claimed in claim 3, characterized by employing a box body (11) containing a U-shaped lamp (13).

5. An appliance as claimed in claim 4, characterized in that a number of air inlets (27) are formed along the edge of a window (18) formed in said box body (11) to channel the air entering said box body (11) inwards.

6. An appliance as claimed in claim 4, characterized in that said box body (11) is fitted inside, at a window (18) formed in said box body (11), with one or more filters (15), each defined by a sheet of glass, for selecting and adapting the light wavelength to the patient's skin by simply replacing the entire box body (11) fitted beforehand with a suitable filter (15).

7. An appliance as claimed in claim 5, characterized in that said filter (15) and said lamp (13) continue to be cooled by said airflow for a given time, even after the treatment session, providing the appliance is connected to the electricity mains.

8. An appliance as claimed in claim 1, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).

9. An appliance as claimed in claim 7, characterized by comprising a pushbutton (47) for controlling electric supply to said electric start circuit (14); and a microswitch (52) connected in series to said pushbutton (47) and for only permitting electric supply to said electric start circuit (14) upon withdrawal of said box body (11) inside said handset (5); said microswitch (52) having a pin (53) cooperating with said plate (35) to follow deformation of the plate and so make or break an electric contact in said microswitch (52).

10. An electric medical appliance for skin treatment, of the type comprising a handset (5) having a lamp (13) for generating, onto an area of a patient's skin, high-energy pulsed light in an ultraviolet to intermediate infrared wavelength range, an electric start circuit (14) for starting said lamp (13), and a filter (15) defined by a sheet of glass and for cutting off light of given wavelengths; characterized in that said handset (5) comprises a rim (24) fixed to a window (18); and in that the maximum opening of said rim (24) may be reduced to adapt to the size of the area for treatment.

11. An appliance as claimed in claim 2, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).

12. An appliance as claimed in claim 3, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).

13. An appliance as claimed in claim 4, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).

14. An appliance as claimed in claim 5, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).

15. An appliance as claimed in claim 6, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).

16. An appliance as claimed in claim 7, characterized in that the handset (5) is fitted inside with a bracket defined by an elastically deformable metal plate (35); bent tabs (38, 41) extending from said plate (35) to enclose and support said box body (11); and said box body (11), when pressed onto the skin, withdrawing inside said handset (5).
